(19) United States
(12) Patent Application Publication
(10) Pub. No.: US 2017/0318881 A1
(43) Pub. Date: Nov. 9, 2017

(54) SHAPE MEMORY MATERIAL GARMENTS

(71) Applicant: ARTHREX, INC., Naples, FL (US)

(72) Inventors: Matthew Fonte, Concord, MA (US); Robert Devaney, Auburndale, MA (US); Matthew Palmer, Medford, MA (US)

(21) Appl. No.: 15/590,138

(22) Filed: May 9, 2017

Related U.S. Application Data
(60) Provisional application No. 62/333,466, filed on May 9, 2016.

(51) Int. Cl. A41D 31/00; A61L 15/26; D03D 15/00; B32B 27/00; C08F 2/01

(52) U.S. Cl. CPC A41D 31/00; A61L 15/26; D03D 15/00; C08F 2/01; B32B 27/00; D10B 2401/046; D10B 2501/02; C08L 2201/12

(57) ABSTRACT

Textiles and garments comprising shape memory materials are disclosed herein. Such garments can include intimate apparel.

### CROSS-REFERENCE TO RELATED APPLICATIONS

This disclosure claims priority to U.S. Provisional Application No. 62/333,466, which was filed on May 9, 2016.

The disclosure of this prior provisional application is incorporated herein by reference in its entirety.

### BACKGROUND

Achieving bare-skin breathability in athletic wear has challenged designers and frustrated wearers for decades. A key obstacle for waterproof and windproof garments is that little or no air can pass through them, i.e., they are not air permeable. Active, perspiring human bodies generate a moist microclimate inside a garment that can make wearing the garment uncomfortable. Dispersion and evaporative cooling through these garments would be extremely beneficial. At very high rates of exertion, moisture from sweat can begin to collect inside a garment, thus raising the potential for either overheating when active or experiencing chills (due to evaporative cooling) when resting. There is a need to improve the breathability of garments that can react to either external climate temperature changes or changes from an increase/decrease in body heat. To address this need, garments 10 (e.g., jackets) can include zippers 12 that are used to open/close vents 14 to help with breathability and allow the bodies to either warm or cool (see, e.g., FIGS. 1A and 1B).

Having to manually open or close a zipper in order to react to a change in body heat is an extra, unnecessary step that can be a nuisance, especially while exercising or being active. Often these vents are opened after body heat has risen, sweat has occurred, and moisture has accumulated. Additionally, some people do not know they are overheating until it is too late and they have already begun to suffer its effects. It would be desirable to create garments (jackets, pants, hats, etc.) that could open or close their pores automatically based on temperature to regulate body heat in real time to prevent moisture from body heat and sweat from accumulating.

### SUMMARY

This disclosure relates to textiles and garments that include combinations of textile materials and shape memory materials. The garments may be personal protective equipment, body temperature regulating apparel, intimate apparel, etc.

An exemplary garment includes, inter alia, a textile material comprising an elastomer, and a nitinol-copper-molybdenum (NiTiCuMo) alloy. The NiTiCuMo alloy is adapted to dilate or contract a pore or channel of the textile material in response to a temperature change. The NiTiCuMo alloy is knitted, woven, sewed, or braided together with the textile material.

An exemplary textile includes, inter alia, a shape memory material adapted to change a physical property in response to a temperature change.

Another exemplary garment includes, inter alia, a textile material and a shape memory material structure incorporated with the textile material. The shape memory material structure is actuable to alter a physical property of the textile material.

The embodiments, examples and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a garment having a zipper for exposing a vent.

FIG. 2 schematically illustrates phase transformations of a shape memory material.

FIG. 3 schematically illustrates Nitinol phase diagrams.

FIG. 4 is an exemplary time-temperature transformation diagram of a Nitinol material.

FIG. 5 schematically illustrates an exemplary shape change of a shape memory material pore.

FIG. 6 schematically illustrates exemplary weaving techniques for creating garments that include shape memory materials with dynamic pores.

FIG. 7 schematically illustrates exemplary knitting patterns that can be made with shape memory materials.

FIG. 8 illustrates a three dimensional spacer fabric made with shape memory materials.

FIG. 9 illustrates an exemplary three dimensional porous structure made with shape memory materials.

FIG. 10 illustrates another exemplary three dimensional porous structure made with shape memory materials.

FIG. 11 illustrates yet another exemplary three dimensional porous structure made with shape memory materials.

FIG. 12 illustrates various exemplary shape memory material mesh structures having dynamic pores.

FIG. 13 illustrates garments that include shape memory material stents or wires inserted into fabric of the garment to allow the fabric to vent.

FIG. 14 schematically illustrates the opening and closing of dynamic pores of a shape memory material embedded in a suspension material.

FIG. 15 schematically illustrates the behavior of a shape memory material wire when actuated by a temperature change.

FIGS. 16A and 16B schematically illustrate the behavior of a corrugated shape memory material structure when actuated by a temperature change.

FIGS. 17A and 17B illustrate closed and open positions of another corrugated shape memory material structure.

FIG. 18 illustrates a window blind as an example of how garments with shape memory materials can behave.

FIG. 19 illustrates exemplary shape memory material tubing.

FIG. 20 illustrates additional exemplary shape memory material tubing.

FIG. 21 illustrates a fleece garment that incorporates shape memory materials.

FIG. 22 illustrates a composite braid that includes a textile material and a shape memory material.

FIG. 23 illustrates intimate apparel that includes shape memory materials.

FIG. 24 illustrates a shape memory mesh for use with intimate apparel.

FIG. 25 illustrates a testing comparison of intimate apparel made of shape memory materials and silicon, respectively.

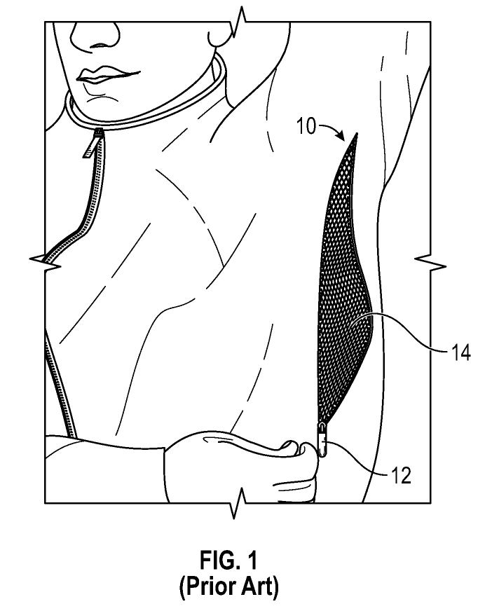

### DETAILED DESCRIPTION

This disclosure describes textiles and garments that include combinations of textile materials and shape memory materials. The garments may be personal protective equipment, body temperature regulating apparel, intimate apparel, etc.

An exemplary garment includes, inter alia, a textile material comprising an elastomer, and a nitinol-copper-molybdenum (NiTiCuMo) alloy. The NiTiCuMo alloy is adapted to dilate or contract a pore or channel of the textile material in response to a temperature change. The NiTiCuMo alloy is knitted, woven, sewed, or braided together with the textile material.

An exemplary textile includes, inter alia, a shape memory material adapted to change a physical property in response to a temperature change.

In a further embodiment, an elastomer pulls or returns a shape memory material to a previous shape and/or properties once cooled below a martensite start temperature.

In a further embodiment, a textile includes one or more materials that are knitted, woven, sewed, or braided together with a shape memory material.

In a further embodiment, a shape memory material is Nitinol.

In a further embodiment, a shape memory material is Nitinol with the addition of Copper or with the addition of Copper and Molybdenum.

In a further embodiment, a shape memory material is a shape memory polymer.

In a further embodiment, a change in a physical property of a textile is the result of dilation or contraction of a pore or channel of the textile.

In a further embodiment, a change in a physical property of a textile is related to the alignment of pores on top of each other to create airflow channels.

In a further embodiment, a textile is a three dimensional spacer fabric.

Another exemplary garment includes, inter alia, a textile material and a shape memory material structure incorporated with the textile material. The shape memory material structure is actuable to alter a physical property of the textile material.

In a further embodiment, a garment is a piece of intimate apparel.

In a further embodiment, a piece of intimate apparel is a bra.

In a further embodiment, a shape memory material structure of a garment is a Nitinol wire or stent.

In a further embodiment, a shape memory material structure of a garment is a mesh patch or stent.

In a further embodiment, a shape memory material structure of a garment is a tube.

In a further embodiment, a textile material and a shape memory material structure of a garment together establish a composite braided structure.

In a further embodiment, a textile material of a garment includes an elastic polymer.

In a further embodiment, a textile material of a garment includes a three dimensional spacer fabric.

In a further embodiment, a textile material of a garment includes a piece of fabric having a plurality of openings or pores.

### Shape Memory Material (SMM)

Nickel-titanium shape memory alloy, known as Nitinol (NiTi), is a functional material whose shape and stiffness can be controlled with temperature. The metal undergoes a complex crystalline-to-solid phase change called martensite-austenite transformation. As the metal in the high-temperature (austenite) phase is cooled, the crystalline structure enters the low-temperature (martensite) phase, where it can be easily bent and shaped. As the metal is reheated above its transition temperature, its original shape and stiffness are restored. Shape memory alloy materials exhibit various characteristics depending on the composition of the alloy and its thermal-mechanical work history. The material can exhibit one-way or two-way shape memory effects. A one-way shape-memory effect results in a substantially irreversible change upon crossing the transition temperature, whereas a two-way shape-memory effect allows the material to repeatedly switch between alternate shapes in response to temperature cycling. Shape memory alloys can recover large strains in two ways: shape memory effect (SME) and pseudoelasticity (i.e., superelasticity (SE)). The NiTi family of alloys can withstand large stresses and can recover strains near 8% for low cycle uses or up to about 2.5% strain for high cycle uses.

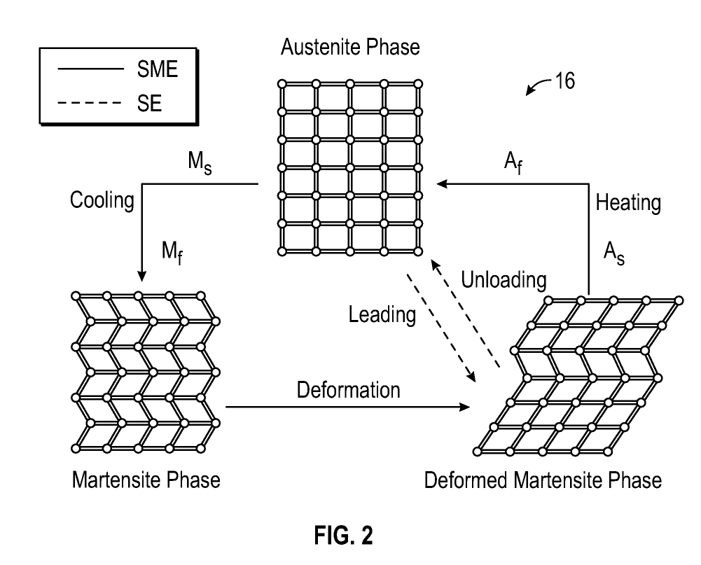

The shape memory alloys, termed as functional materials, show two unique capabilities: shape memory effect (SME) and superelasticity (SE), which are absent in traditional materials. Both SME and SE largely depend on the solid-solid, diffusionless phase transformation process known as martensitic transformation (MT) from a crystallographically more ordered parent phase (austenite) to a crystallographically less ordered product phase (martensite). As shown schematically in FIG. 2, a phase transformation 16 of a shape memory material (from austenite to martensite or vice versa) is typically marked by four transition temperatures: Martensite finish (M_{f}), Martensite start (Mₛ), Austenite finish (A_{f}), and Austenite start (Aₛ) (where M_{f}<Mₛ<Aₛ<A_{f}). Thus, a change in the temperature within Mₛ<T<Aₛ induces no phase change and both martensite and austenite may coexist within M_{f}<T<A_{f}. The phase transformations may take place depending on changing temperature (SME) or changing stress (SE).

### Aging of Shape Memory Alloy

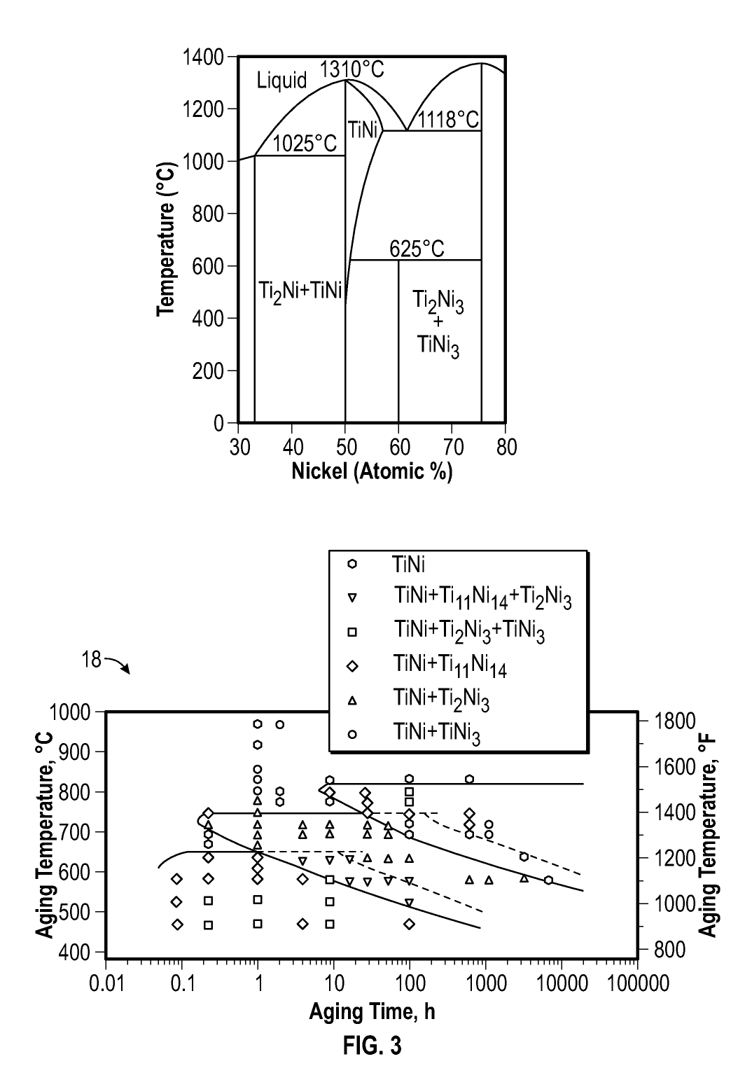

It may be desirable for the A_{f} temperature to be relatively close to body temperature (37° C.). In the case of Nitinol, for example, the starting material may include an A_{f} around body temperature; however, the transformation temperatures may change as a result of any cold work and heat treatment steps used to manufacture the final product. It is possible to return the Nitinol to its fully annealed state by heating it to 800° C. to 850° C. for about 15 to about 60 minutes. This generally erases all thermomechanical processing. Subsequently, the A_{f} temperature can be reset by aging the material. The A_{f} temperature may be affected by the exact matrix composition. As can be seen on the Nitinol phase diagram 18 of FIG. 3, as the aging temperature and time increases, nickel rich precipitation reactions occur. These changes may affect how much nickel is in the NiTi lattice structure. By reducing the amount of nickel in the matrix, aging increases the transformation temperature.

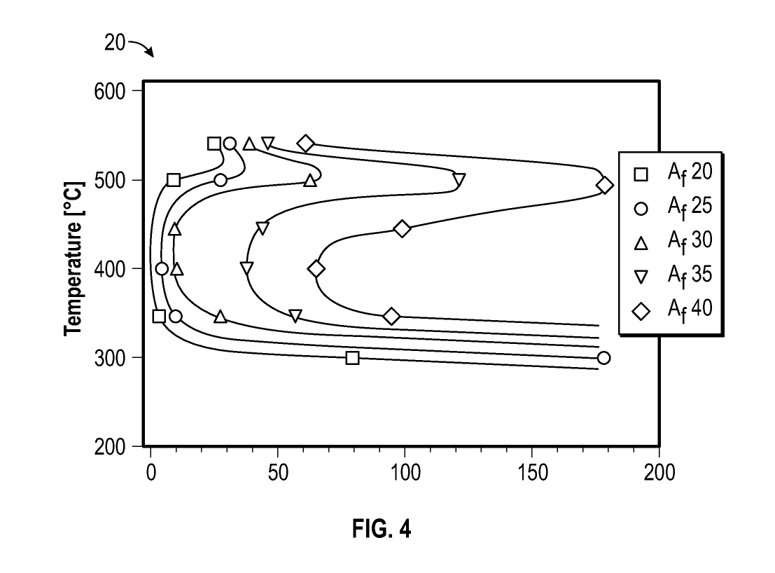

It is possible to read a TTT (time-temperature transformation) diagram 20 (see FIG. 4) to determine at what temperature and for what period of time to age the Nitinol material to achieve an appropriate A_{f}. As seen in the TTT diagram 20, aging the Nitinol material at 400° C. for approximately 30 minutes results in an A_{f} close to 37° C. In an embodiment, the exact A_{f} temperature can be measured using a differential scanning calorimeter.

### Shape Memory Effect (SME)

For T>A_{f}, the shape memory alloy is in the parent austenite phase with a particular size and shape. Under stress free conditions, if the shape memory alloy is cooled to any temperature T<M_{f}, martensitic transformation (MT) occurs as the material converts to product martensite phase. MT is basically a macroscopic deformation process, though actually no transformation strain is generated due to the so-called self-accommodating twinned martensite. If a mechanical load is applied to this material and the stress reaches a certain critical value, the pairs of martensite twins begin ‘detwinning’ (conversion) to the stress-preferred twins. The ‘detwinning’ or conversion process is marked by the increasing value of strain with insignificant increase in stress. The multiple martensite variants begin to convert to single variant, the preferred variant determined by alignment of the habit planes with the axis of loading. As the single variant of martensite is thermodynamically stable at T<Aₛ, upon unloading there is no reconversion to multiple variants and only a small elastic strain is recovered, thus leaving the materials with a large residual strain (apparently plastic). Next, if the deformed shape memory alloy is heated above A_{f}, the shape memory alloy transforms to parent phase (which has no variants), the residual strain is fully recovered, and the original geometric-configuration is recovered. This happens as if the material recalls from ‘memory’ its original shape before the deformation and fully recovers. Therefore, this phenomenon is termed as shape memory effect (one-way SME). However, if some end constraints are used to prevent this free recovery to the original shape, the material generates large tensile recovery stress, which can be exploited as actuating force for active or passive control purposes. Shape memory material coatings can be processed via SME.

### Superelasticity (SE)

The second feature of shape memory alloys is pseudoelasticity. The superelastic shape memory alloy has the capability to fully regain the original shape from a deformed state when the mechanical load that causes the deformation is withdrawn. For some superelastic shape memory materials, the recoverable strains can be on the order of 10%. This phenomenon, termed as the pseudoelasticity, superelasticity (SE), is dependent on the stress-induced martensitic transformation (SIMT), which in turn depends on the states of temperature and stress of the shape memory material. To explain the SE, it is assumed that the shape memory material has been entirely in the parent phase (T>A_{f}) and is mechanically loaded. Thermodynamic considerations indicate that there is a critical stress at which the crystal phase transformation from austenite to martensite can be induced. Consequently, the martensite is formed because the applied stress substitutes for the thermodynamic driving force usually obtained by cooling for the case of SME. The load, therefore, imparts an overall deformation to the SMA specimen as soon as a critical stress is exceeded. During unloading, because of the instability of the martensite at this temperature in the absence of stress, again at a critical stress, the reverse phase transformation starts from the SIM to parent phase. When the phase transformation is complete, the shape memory material returns to its parent austenite phase. Therefore, superelastic SMA shows a typical hysteresis loop (known as pseudoelasticity or superelasticity) and if the strain during loading is fully recoverable, it becomes a closed one. It should be noted that SIMT (or reverse SIMT) are marked by a reduction of the material stiffness. Usually the austenite phase has much higher Young's modulus in comparison with the martensite phase.

Nitinol cardiovascular stents, orthodontic wires, and other commercially available wire and thin wall tubing products utilize the material's superelastic characteristics. The setting of the material's A_{f} temperature is typically set in relation to body temperature. Stress induced martensite transformation (SIMT) may be used to collapse the products' diameter to facilitate minimally invasive insertion into a body. The material is expanded in the body once free from a constrained/stressed state to desirably apply a long-term compression of tissues or bones.

### Creation of Textiles with Dynamic Pores made from Shape Memory Materials (SMM)

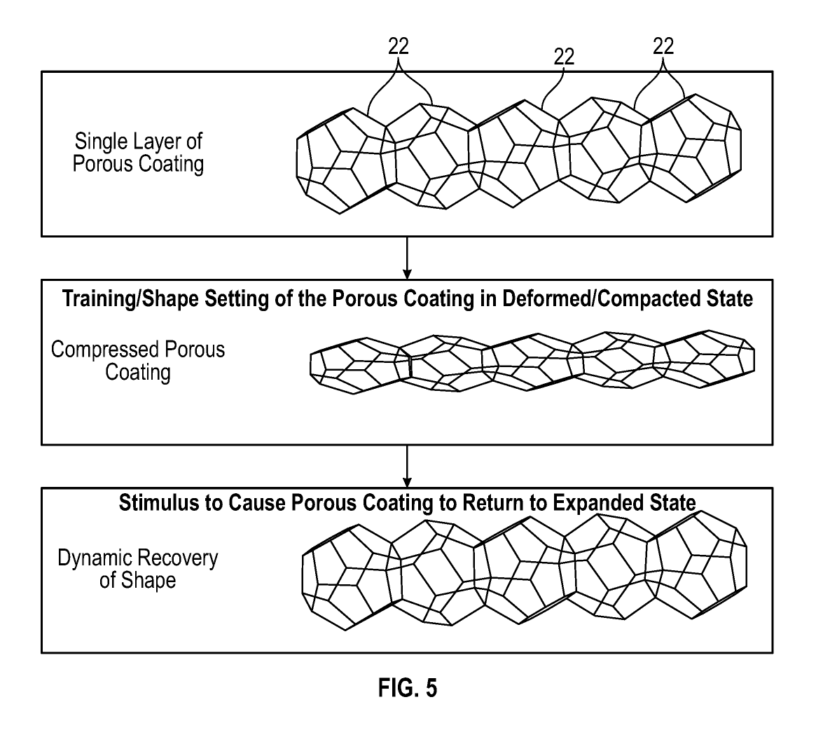

Dynamic pores of a shape memory material can exhibit one-way or two-way shape memory effects and can exhibit SE or SME characteristics. Referring to FIG. 5, a shape memory material may include a plurality of pores 22. The pores 22 can change shape based on SE and SME. For example, the pores 22 can move to a compressed, deformed, or compacted state under compression and can recover to an expanded state when the compression force is removed.

In an embodiment, textiles may be made from SMM using any traditional knitting, weaving, sewing, and/or braiding techniques. When the SMM warms to above the Aₛ temperature, the material changes shapes and pores are opened to allow body heat to vent out of the garment. When the material is later cooled below its Mₛ temperature, it softens and elastomers (i.e. Spandex or other elastic polymers) can elastically pull the SMM back to its original position, effectively closing the pores. SME can be used to actuate the SMM to open its pores, and elastomers are used to pull or actuate the SMM to return to its original shape with the closed pores.

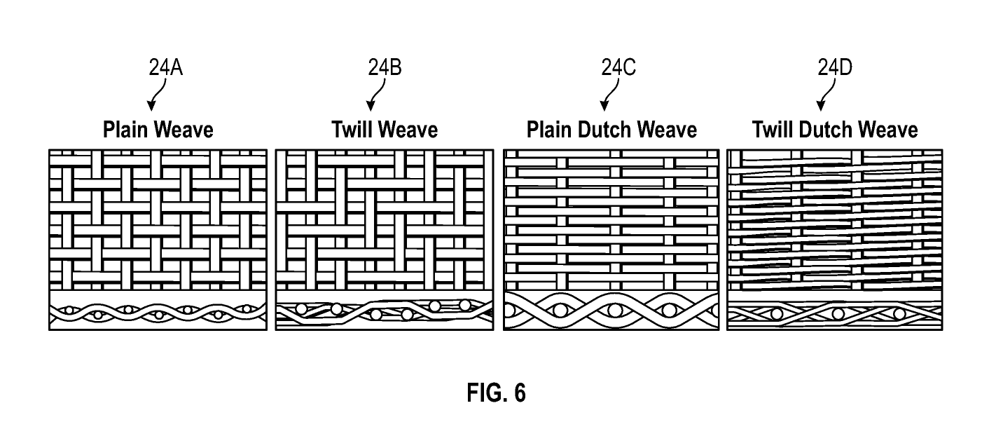

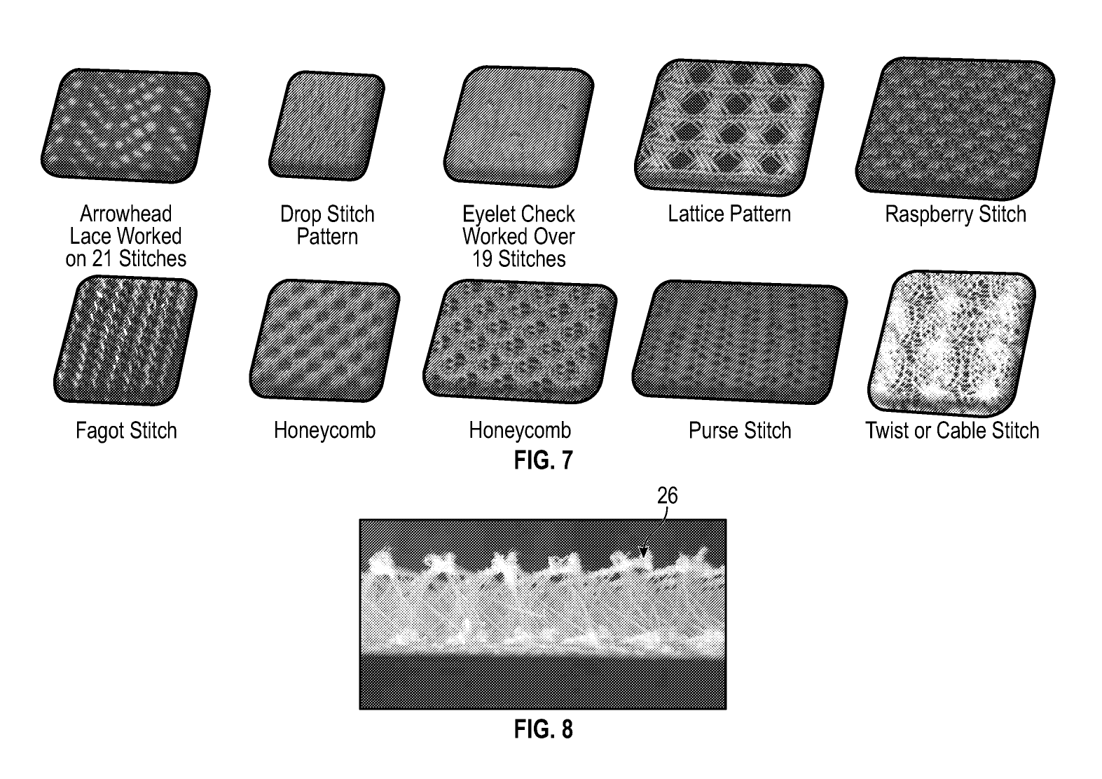

In another embodiment, the pores of the SMM are created in garments using various weaving techniques. There are several different types of weaving techniques, including but not limited to, plain weave 24A, twill weave 24B, plain dutch weave 24C, and twill dutch weave 24D. The weaving techniques are schematically illustrated in FIG. 6. Additionally, knitting either warp or weft, single bar, or multibar allowes for many different patterns and structures to be created. In yet another embodiment, as shown in FIG. 7, a variety of stitching patterns can be used to create garments that include SMM.

The above techniques afford a large variety of different geometries and structures. It is possible to create mono-layer and multi-layer shape memory materials, as well as tubular structures. It is also possible to create structures with varying widths and thicknesses within the same structure. Multiple layers of structures can be laminated on top of one another to create a three-dimensional structure, or they can be sewn, knit, or woven directly into a three dimensional structure. By layering multiple sheets of materials with different pore sizes and geometries on top of one another, a dynamic three-dimensional fabric with a complex interconnected network of pores can be created. The dynamic SMM pores can be constructed as a single or multilayered sheet that can be combined into the rest of a garment's textile.

The various knitted and woven structures can be layered on each other. In an embodiment, the knitted/woven structures are layered to form a three dimensional knit spacer fabric 26 made with SMMs (see FIG. 8). When two or more layers of a SMM are offset from one another, the two or more layers can create a closed combined layer. Alternatively, if the layers are aligned on top of one another in the same orientation, the pores between the two layers can be formed to allow ventilation in the garment. For example, if the top layer is angled at 15° degrees offset from the bottom layer, there might not be a line of sight between the two layers. On the other hand, the layers could be turned or rotated to allow the multi layers to open holes or to establish a line of sight through the composite structure. The knitted or woven structures could be made of SMM. When the SMM warms to above the Aₛ temperature, one or more of the layers rotate to allow pores to open between the layers to allow body heat to vent out of the garment. When the material is later cooled below its Mₛ temperature, the layer softens to its martensite condition and an elastomer (i.e., Spandex or other elastic polymers) can elastically pull the SMM material back to its original position, effectively closing the pores between the composite layer. SME can be used to actuate the SMM layer to open pores between the two more layers, and elastomers can be used to pull or actuate the SMM layers to return to their original orientation to close the pores. The knitted or woven structures can be made of polymers that use SMM fibers to push or pull one layer to rotate to open pores or close pores in the multilayer (composite) structure.

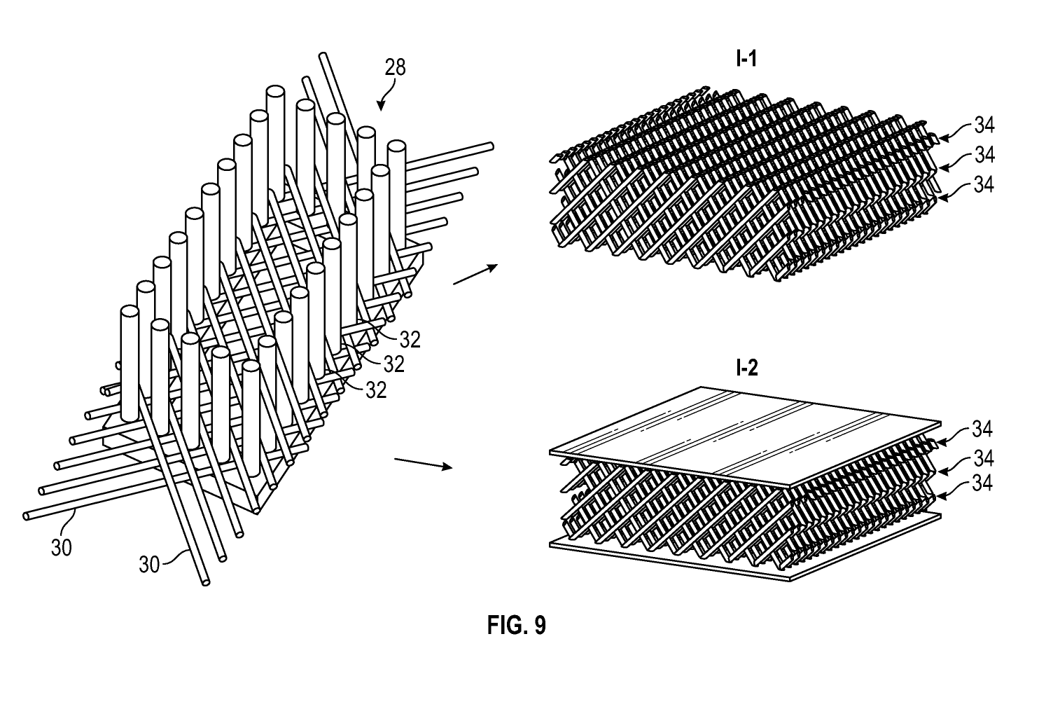

It is also possible to create a dynamic porous structure using non-traditional textile manufacturing methods. Referring to FIG. 9, for example, a jig 28 may be used to weave wires 30 and/or tubes 32 in various patterns, thus building multiple layers 34 one on top of another (see insets I-1 and I-2 of FIG. 9). The overall structure can then be sintered to fuse the layers 34 together, if so desired.

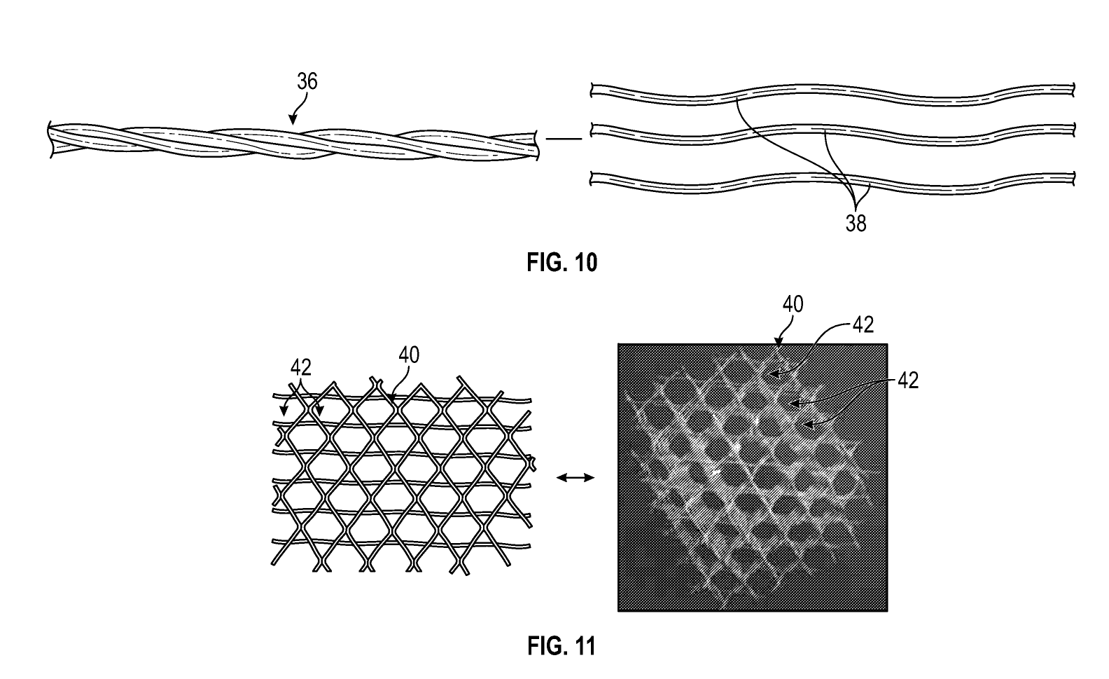

Another exemplary method for weaving a three dimensional, SMM porous structure is a modified Kagome weave 36, which is shown in FIG. 10. In an embodiment, the Wire Woven Bulk Kagome (WBK) is assembled from continuous helical wires 38 systematically arranged in six directions.

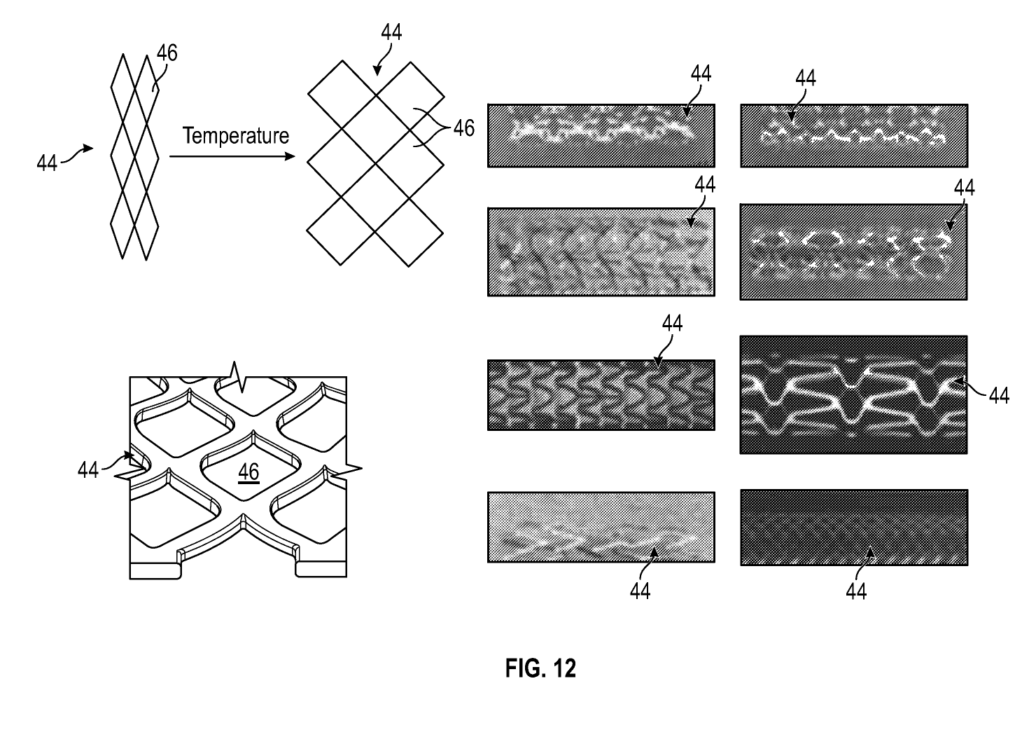

Yet another exemplary method of creating an SMM porous structure is to create a diamond shape mesh 40, as shown in FIG. 11. In the martensitic condition, the mesh 40 is closed flat, and at a hotter temperature the material flips to austenite and pores 42 of the diamond like mesh 40 are opened to enhance breathability. FIG. 12 illustrates additional meshes 44 having pores 46 that are closed at one temperature and open at a second, warmer temperature. The SMM meshes can take various sizes, shapes, and configurations within the scope of this disclosure.

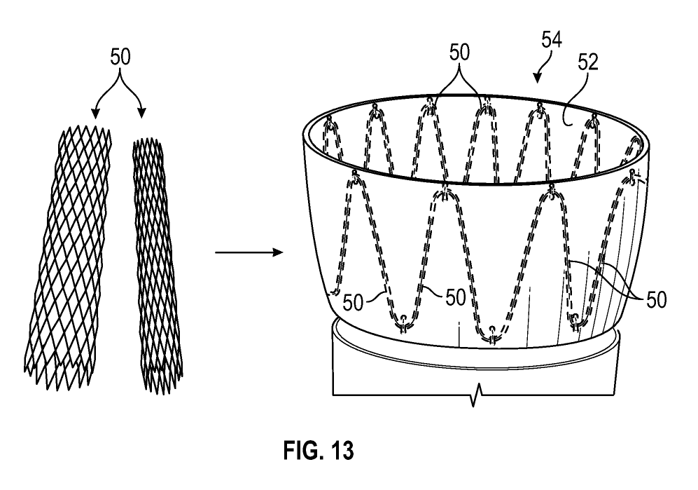

Referring now to FIG. 13, SMM (e.g., NiTi) stents 50 (or wires) can be incorporated into a piece of fabric 52 of a garment 54. The SMM stents 50 act as elastic that has dynamic pores that can bent and/or expanded to open and vent. At a first, colder temperature, the SMM stent 50 has a closed diameter with closed pores, and at a second, hotter temperature the SMM stent 50 opens creating larger pores for the garment 54 to vent. Nitinol wire can be woven through the garment's fabric to help pull the pores open at various temperatures. When the SMM material warms to above the Aₛ temperature, the material changes shapes and the pores are pulled opened to allow body heat to vent out of the garment 54. When the material is later cooled below its Mₛ temperature, it softens and elastomers (i.e. Spandex or other elastic polymers) can elastically pull the SMM back to their original position, effectively closing the pores. Shape Memory Effect (SME) may be used to actuate the SMM to open the pores and elastomers may be used to pull or actuate the SMMs to return to their original shape with the closed pores.

The opening and closing of pores could stretch a garment's fabric and lead to a poor or uncomfortable fit. To address this issue, an SMM structure can be embedded in a section of fabric that includes a higher elasticity than other sections of fabric of the garment. This would act as a suspension and isolate the garment from the pore actuation.

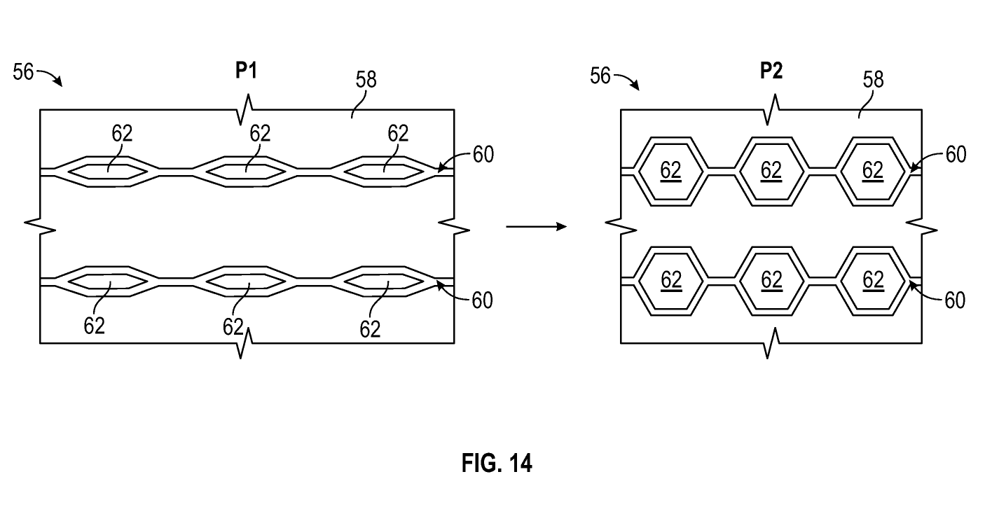

For example, in an embodiment depicted in FIG. 14, a garment 56 includes a suspension material 58 and one or more SMM structures 60. The SMM structures 60 may be embedded in the suspension material 58. The SMM structures 60 may include dynamic pores 62. The dynamic pores 62 may change from a more closed position P1 to a more open position P2 in response to a temperature change.

In yet another exemplary embodiment, as depicted in FIG. 15, SMM structures 64 can be laminated between one or more fabricate layers 66 of a traditional pored fabricate to create a garment 69. The SMM structures 64, when actuated by a change in temperature, can change shape and either cause pores 68 of the fabricate layers 66 to open or close. In an embodiment, the SMM structures 64 are sinusoidal shaped wires that straighten when heated, thus opening the pores 68 of the fabricate layers 66.

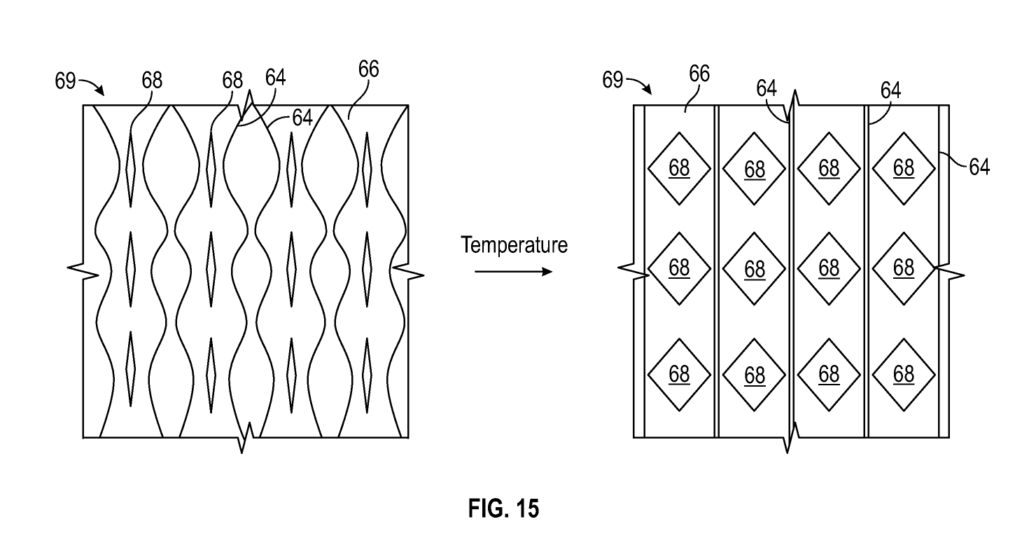

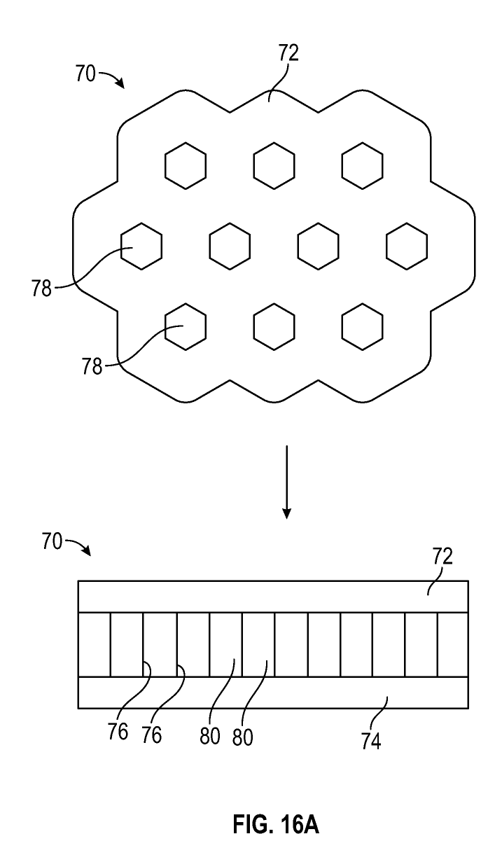

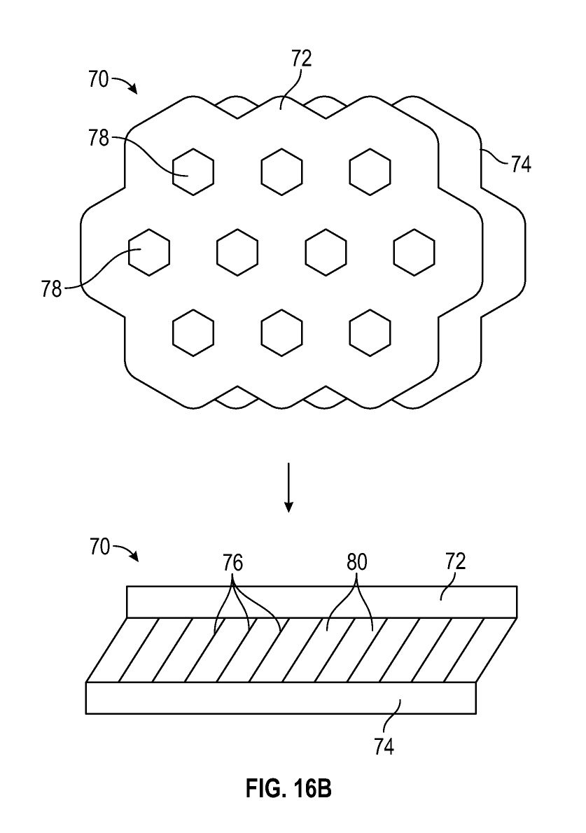

3D knitting technology also represents a way to create dynamic pores in a garment. As shown in FIGS. 16A and 16B, spacer fabrics 70, which are a type of 3D knit structure, are composed of a top face 72, a bottom face 74, and fibers 76 that extend therebetween. It is possible to create this type of structure using SMMs. At one temperature (e.g., a warmer temperature), as shown in FIG. 16A, openings or pores 78 in the top face 72 are aligned on top of openings or pores 78 in the bottom face 74, thus creating channels 80 for allowing cooling air to pass through the garment (see FIG. 16A). In an embodiment, the pores 78 are hexagonal shaped. At a different temperature (e.g., a colder temperature), the pores 78 on the top face 72 can be offset from the pores 78 on the bottom face 74, thus closing the channels 80 for blocking airflow through the garment (see FIG. 16B).

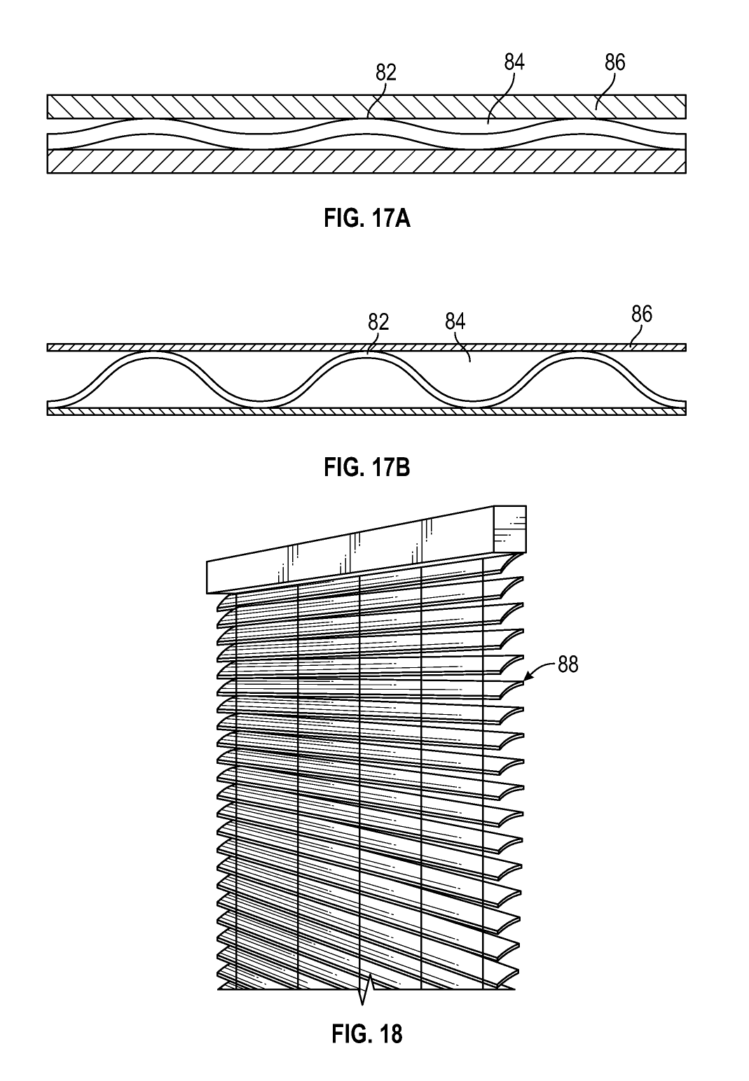

A similar effect can be obtained by using a corrugated SMM structure 82 placed inside a channel 84 formed in a traditional textile 86. At one temperature, the SMM structure 82 will be generally flat, thus pulling the channel 84 of the textile 86 closed (see FIG. 17A). At a different temperature, the SMM structure 82 will take a 3D corrugated form and push the channel 84 further open, thus allowing airflow through the garment (see FIG. 17B).

Importantly, textiles with dynamic pore structures do not need to be manufactured entirely from SMMs. They can be manufactured using traditional textile materials, and incorporate SMMs selectively to actuate the physical property changes in the porous structure. The resulting structures are aged at one temperature and then deformed to a different structure. At the transition temperature, the structure will resume the original structure configuration.

In an embodiment, SMM fibers/wires can be made like Venetian Blinds 88 (see, e.g., FIG. 18) to open and close at various temperatures via Shape Memory Effect. The fibers can actuate open when hot (austenite) and an elastomer or spandex can pull the blinds closed when cooled to (martensite). The blinds can open up and down or rotate torsionaly left to right. The blinds can be made of SMMs, or just the “strings” or fibers that pull the blinds can be made of SMMs.

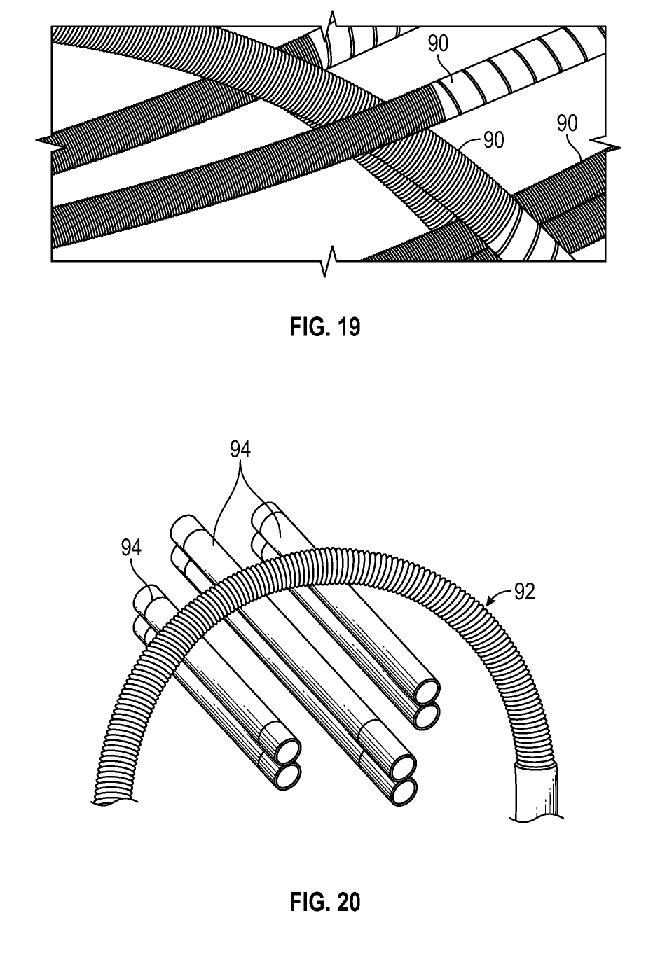

Another exemplary method of allowing pores to open in garments is to have a series of tubular channels made of SMM reinforced plastic tubing sewn into the garment. When the SMM is cool, the reinforced plastic tubing includes a small diameter. When the SMM becomes warmer, the SMM will flip from martensite to austenite, radially expand through Shape Memory Effect, and circumferentially expand the reinforced plastic tubing. Pores in the plastic tubing will be stretched or dilated (opened) allowing heat to escape. The plastic reinforced tubing with holes or pores can allow air to pass freely (cross-sectionally) therethrough while venting/cooling longitudinally through the tubes similar to a chimney Should rain or other moisture enter the open pores, the tubing can act as a drainage pipe funneling the water to the bottom and out of the tubing. The plastic tubing can also be made out of Shape Memory Polymers, with or without the reinforced SMM. When the SMM cools again, the SMM will revert from austenite to martensite, or its weaker phase. The stretched plastic tubing will be engineered to exert enough force to collapse the SMM to its smaller diameter shape and close the pores, thus insulating them. Exemplary SMM reinforced plastic tubing 90 is illustrated in FIG. 19.

There are three basic types of reinforcement tubing: braiding, spiral or coil, and linear members. With braiding, the braid angle and percent coverage are important specifics, as are the size, shape, and tensile strength of the reinforcing material. Braid angle is measured from the longitudinal axis of the tube. This means that a 30° braid angle is closer to parallel with the axis than it is perpendicular. Changing the braid angle changes the flexibility and torque response of the tube. Typically, a lower angle creates a stiffer tube that can deliver more torque and reduce stretching, while a higher angle creates a more flexible kink-resistant tube with somewhat lower torque transmission. Braiding machines are available that can automatically toggle between several braid rates during the run. This creates a product with different braid flexibility between the proximal and distal sections of a shaft without the expense or risk of a molded joint. Higher percent coverage can also add to kink resistance, torque transmission, and pressure resistance. However, if the coverage is too high it interferes with layer bonding, which in turn defeats the performance advantages of the reinforcing material.

Spiral reinforcement allows for high (almost perpendicular) angles. High angles take advantage of the reinforcing wire's tight helical configuration using its hoop strength to provide good kink and crush resistance. However, a high-angle spiral design provides almost no torque transmission and will not prevent linear stretching of the tube. When using spiral reinforcements, important characteristics include tensile strength, material, size, and cross section of the reinforcing element, durometer of plastic compounds, and wall thickness. Continuous spiral reinforced tube manufacturing is more limited in availability than continuous braid reinforced tube. This is because of cost and availability of specialized equipment required to manufacture this type of reinforced tubing.

Linear reinforcement provides excellent stretch resistance but limits flexibility depending on the number and location of reinforcing members. It is also possible to combine braided or spiral reinforcing with linear reinforcing elements to produce a hybrid design. Reinforcement material, tensile strength, size, and placement of the elements are critical aspects with linear reinforcing.

There are primarily two ways to manufacture thermoplastic reinforced tubing. The first is continuous-layer processing, and the second is called component reflow. Continuous-layer processing uses sequential extrusion and reinforcing steps using long lengths of material. Special extrusion equipment and processing conditions bond the extrusion layers through the reinforcing component. Typical runs are around 2,000 to around 20,000 feet. Generally, just one material is extruded during each run, although some manufacturers have equipment to extrude different materials intermittently during a single run.

The reflow method produces one unit at a time. In an embodiment, an operator takes a pre-made, cut length extrusion and braid components and layers them by hand onto a solid metal mandrel. Heat-shrink tubing slides over the assembled parts and the whole unit is baked in an oven. The heat shrink applies circumferential compression to the polymer layers while transferring heat to the lower melt temperature materials on the mandrel, thereby laminating them together. The advantage of this method is that it combines several different materials longitudinally and “reflows” them together. Also, the reinforcing member can be started and stopped in discrete positions along the shaft. Because this method involves a large amount of handwork, the cost per unit may be significantly higher than parts made with the continuous-layer extrusion process.

In another garment embodiment, an SMM coil, braid, or stent 92 can be inserted inside a plastic tube 94 (see, e.g., FIG. 20). A reinforced tube made with a Shape Memory Material has a composite structure. The polymer layers and reinforcing materials are formed into one structure that may exhibit different performance characteristics from the individual materials. If the SMMs actuating force when austenitic is greater than the composite structure it will expand the tube. If the composite structure is stronger than the SMM in its martensite condition, the composite structure will collapse the tube's diameter. Performance characteristics using a number of different design combinations are required to optimize the ability to expand at one temperature and collapse a second colder temperature. The style and design of the reinforcement and the thicknesses of the polymer layers can be varied for specific performance characteristics. For example, the tube diameter can increase through shape memory effect to open pores and vent a garment. If there are not any pores in the tube, the expansion of the tube can create air pockets/channels to affectively create insulation layers to keep warm.

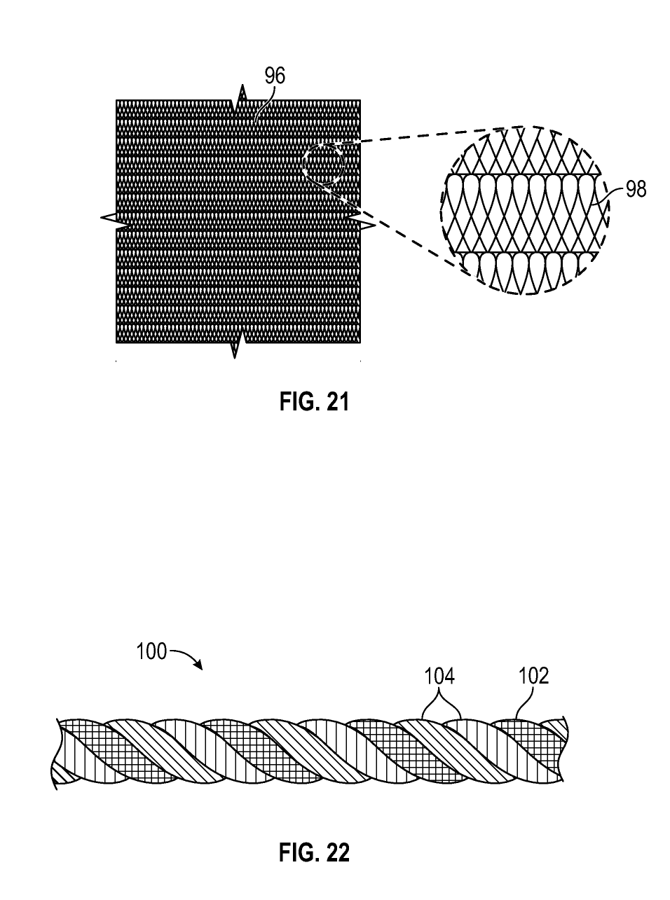

In another embodiment, a garment may include fleece materials 96 that incorporate SMMs 98 (e.g., SMM wires) (see, e.g. FIG. 21). Fleece that incorporates SMMs can used in two different ways. For example, the SMMs can be in the superelastic condition so that regardless of temperature, the fleece is always in a low density, “fluffy” state. In another embodiment, the SMMs can be used for their shape memory effect. At lower temperatures the SMMs stand erect, thus fluffing the fleece material. At higher temperatures, the SMMs lay flat, thereby decreasing the volume of fleece material and decreasing its insulating capacity. The use of SMM wires to keep a material in the low density state can also be applied to other materials, such as down insulation. When wet, down becomes compressed and loses its insulating properties. Down insulation that incorporates SMM can be maintained in the “fluffed” state even when wet.

In yet another embodiment, custom fibers can be created that incorporate SMMs that can then be used in traditional knitting, weaving, or braiding processes. An exemplary method of doing this is to braid or twist a composite structure 100, where one of the fibers includes a SMM 102 and the other fiber or fibers are traditional textile materials 104 (see FIG. 22). One of the fibers in the composite structure 100 may also be an elastomer. In such an embodiment, the elastomer can be used to counter the movement generated by the SMM during its phase transformation. The SMM can change shape upon heating, and when cooled, the elastomer can cause the material to return to its original shape.

### Exemplary Shape Memory Materials

In an embodiment, a shape memory alloy may include Nitinol (Nickel Titanium alloy). However, Nitinol has a relatively large hysteresis loop, which means there can be a large delta in the Martensite Start (Mₛ) and the Austenite Start (Aₛ) temperatures. Thus, the temperature at which the SMM actuates to open pores is high and the temperature that the SMM cools to form Martensite alloying the elastomer to pull it close is much lower, perhaps 40° Fahrenheit between the Mₛ and Aₛ. In order to close this delta in actuation and re-sizing while martensite, the Nitinol alloy can include a third metal, such as Copper, to close this delta. For example Ti—Ni—Cu alloys has been known to be very attractive in applications for an actuator, since these alloys include a relatively large transformation elongation (2.5-3.2%) and small hysteresis (4-12 K). A forth metal, such as Molybdenum, can be added to produce yet another alloy (NiTiCuMo), which can also be used to close the delta between Mₛ and Aₛ. For example, Ti-34.7Ni-15Cu-0.3Mo alloys have a transformation temperature as shown below in Table 1:

TABLE 1
Transformation temperatures of Ti—Ni—Cu—Mo alloys.

| | Transformation temperatures (K) | | | | | | | |
|---|---|---|---|---|---|---|---|---|
| Cu-content | Ms′ | Mf′ | As′ | Af′ | Ms | Mf | As | Af |
| 5 | 277 | — | 266 | 280 | 265 | 225 | 229 | — |
| 10 | 285 | 268 | 263 | 300 | | | | |
| 15 | 294 | 290 | 295 | 299 | | | | |
| 20 | 302 | 296 | 300 | 306 | | | | |

### Creation of Intimate Apparel Using Shape Memory Materials

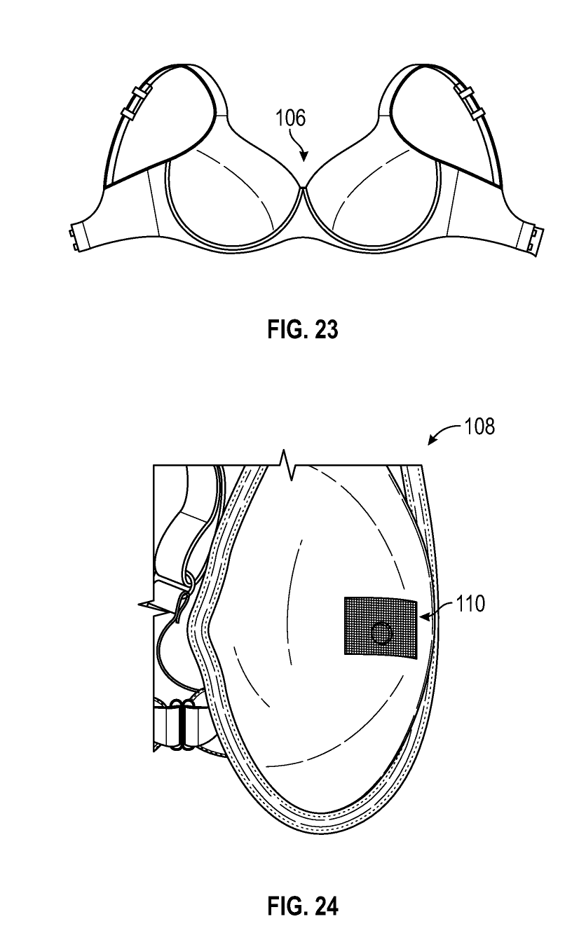

The unique qualities of SMMs allow for innovative uses of SMM in garments such as intimate apparel. In bra design (see bra 106 of FIG. 23, for example), two common issues are nipple concealment and breast shaping. SMMs, such as those used in the formats previously described such as a mesh or a 3D spacer fabric, can address both of these issues. SMM in all forms can be shape set or molded into the unique geometries required for the construction of bras. 3D spacer fabric constructed of super elastic SMM is a dynamic material that can be used for both shaping and padding in bras. SMM also addresses the problem of nipple concealment by both sheltering the nipple and providing an inward force that prevents the nipple from protruding outward. SMMs can also be used to conceal the nipple in shear bras. 2D SMM meshes can shape set into 3D structures, such as hemispheres, that provide similar padding and nipple concealment to 3D spacer fabrics. Transition temperatures for the SMM can be set to change by the heat generated by a hair dryer, for example. Thus, the wearer can use a hair dryer (or other suitable heating device) to heat the SMM and alter the shape of the undergarment. Heating may cause the SMM to alter its shape to enhance or limit the appearance of cleavage. Additionally, many women's breasts are different sizes. Current undergarments have the same size cup for the left and right breast. SMMs can be used to adjust the fit of a single undergarment to support each breast independently.

Additionally, SMM can be used to enhance the function of elastic materials. By creating a flat braid of SMM wire, for example, a material is created that acts similarly to elastic; however, unlike elastic, SMM will not lose it stretchiness over time. To enhance the feel of the material, the SMM can be embedded in traditional elastic to enhance the tactile feel of the material.

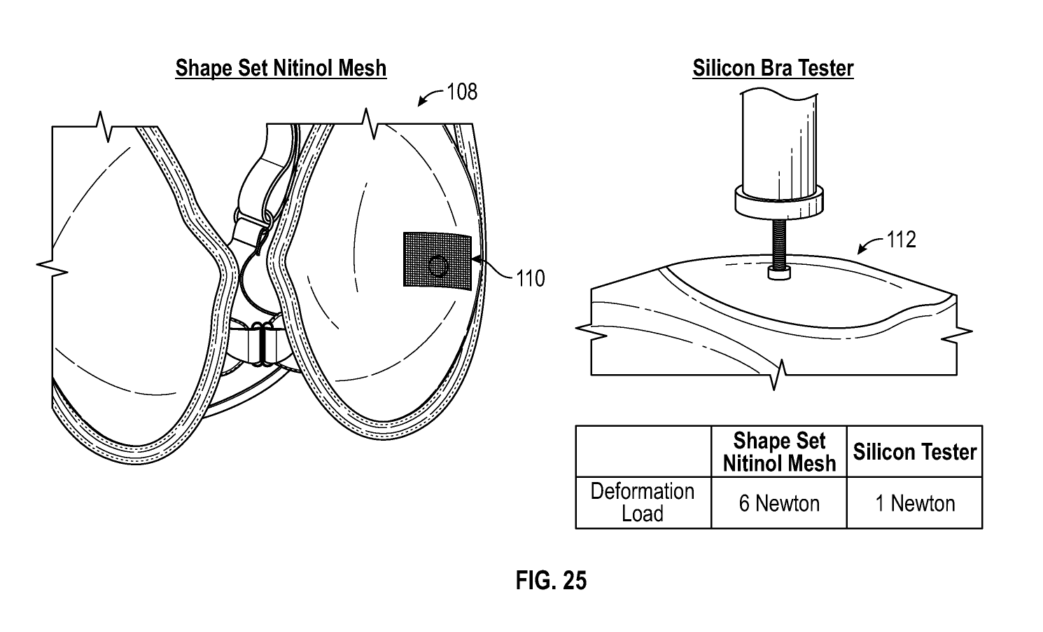

Referring to FIG. 24, another exemplary bra 108 may include an SMM patch 110 for concealing a nipple within the bra 108. In an embodiment, the SMM patch 110 is a mesh patch that has been shape set into a hemispherical geometry that extends outward from the inside of the bra 108. The SMM patch 110 can thus exert an inward force that prevents the nipple from protruding outward of the bra 108. FIG. 25 schematically illustrates a testing comparison of the bra 108 having the SMM patch 110 and a silicon bra 112. As depicted, the deformation load of the SMM patch 110 is higher than the silicon bra 112.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A garment, comprising:
a textile material comprising an elastomer; and
a nitinol-copper-molybdenum (NiTiCuMo) alloy;
wherein the NiTiCuMo alloy is adapted to dilate or contract a pore or channel of the textile material in response to a temperature change; and
wherein the NiTiCuMo alloy is knitted, woven, sewed, or braided together with the textile material.

2. A textile, comprising:
a shape memory material adapted to change a physical property in response to a temperature change.

3. The textile as recited in claim 2, comprising an elastomer to pull or return the shape memory material to a previous shape and/or properties once cooled below a martensite start temperature.

4. The textile as recited in claim 2, comprising one or more other materials that are knitted, woven, sewed, or braided together with the shape memory material.

5. The textile as recited in claim 2, where the shape memory material is Nitinol.

6. The textile as recited in claim 2, wherein the shape memory material is Nitinol with the addition of Copper or with the addition of Copper and Molybdenum.

7. The textile as recited in claim 2, wherein the shape memory material is a shape memory polymer.

8. The textile as recited in claim 2, where the change in the physical property is the dilation or contraction of a pore or channel of the textile.

9. The textile as recited in claim 2, wherein the change in the physical property is related to the alignment of pores on top of each other to create airflow channels.

10. The textile as recited in claim 2, wherein the textile is a three dimensional spacer fabric.

11. A garment, comprising:
a textile material; and
a shape memory material structure incorporated with the textile material, wherein the shape memory material structure is actuable to alter a physical property of the textile material.

12. The garment as recited in claim 11, wherein the garment is a piece of intimate apparel.

13. The garment as recited in claim 12, wherein the piece of intimate apparel is a bra.

14. The garment as recited in claim 11, wherein the shape memory material structure is a Nitinol wire or stent.

15. The garment as recited in claim 11, wherein the shape memory material structure is a mesh patch or stent.

16. The garment as recited in claim 11, wherein the shape memory material structure is a tube.

17. The garment as recited in claim 11, wherein the textile material and the shape memory material structure together establish a composite braided structure.

18. The garment as recited in claim 11, wherein the textile material includes an elastic polymer.

19. The garment as recited in claim 11, wherein the textile material includes a three dimensional spacer fabric.

20. The garment as recited in claim 11, wherein the textile material includes a piece of fabric having a plurality of openings or pores.
